(19) United States
(12) Patent Application Publication
(10) Pub. No.: US 2022/0072192 A1
(43) Pub. Date: Mar. 10, 2022

(54) BIOCOMPATIBLE CARBOXYMETHYLCELLULOSE MATRIX (BCM) FOR HEMOSTASIS, TISSUE BARRIER, WOUND HEALING, AND COSMETOLOGY

(71) Applicant: LifeSciencePLUS, Inc., Mountain View, CA (US)

(72) Inventors: Guiting Lin, San Francisco, CA (US); Vicky Feng, Mountain View, CA (US)

(21) Appl. No.: 17/492,618

(22) Filed: Oct. 3, 2021

Related U.S. Application Data
(63) Continuation of application No. 15/766,174, filed on Apr. 5, 2018.
(60) Provisional application No. 62/238,676, filed on Oct. 7, 2015.

(51) Int. Cl. A61L 15/28; A61L 15/42; A61L 15/44

(52) U.S. Cl. CPC A61L 15/28; A61L 15/425; C08L 1/286; A61L 2400/04; A61L 2300/232; A61L 15/44

(57) ABSTRACT

The invention provides novel hemostasis, tissue barriers, wound healing and cosmetology materials based on biocompatible carboxymethylcellulose, and methods for their preparation and use thereof.

### PRIORITY CLAIMS AND RELATED PATENT APPLICATIONS

This application claims the benefit of priority from U.S. Provisional Application Ser. No. 62/238,676, filed on Oct. 7, 2015, the entire content of which is incorporated herein by reference in its entirety.

### TECHNICAL FIELDS OF THE INVENTION

The invention generally relates to hemostats and wound care. More particularly, the invention relates to novel hemostasis, tissue barriers, wound healing and cosmetology materials based on biocompatible carboxymethylcellulose, and methods for their preparation and use thereof.

### BACKGROUND OF THE INVENTION

Wound and burn healing processes are intricate, complex and dynamic skin and body tissue repair processes. In healthy skin, the epidermis and dermis form a protective barrier. Due to damage and death of tissue at the site of the wound or burn, wounds and burns are susceptible to infection by microorganisms, such as bacteria and fungi. Microbial infection slows or prevents the healing process and can lead to a localized or systemic infection. The wound and burn healing processes are not only complex but also fragile, and are susceptible to disruption or breakdown leading to slowing or non-healing and chronic wounds. Timely and proper wound and burn care boosts and speeds wound and burn healing and reduce risk of re-injury or infection.

Hemostasis is a process that causes bleeding to stop by keeping blood within a damaged blood vessel. Bleeding can result from a variety of unintentional causes (e.g., injuries, diseases) as well as variety of intentional causes (e.g., surgeries, blood tests). Hemostasis is the first stage of wound healing.

Certain deficiencies exist with respect to conventional hemostasis devices for providing proper barrier and encouraging tissue repair. An ongoing need remains for novel and improved hemostasis, tissue barriers and wound and burn healing materials and devices.

### SUMMARY OF THE INVENTION

The invention is based in part on the discovery of unique and much improved effect of hemostasis, tissue barrier, wound and burn healing and cosmetology of certain biocompatible carboxymethylcellulose-based materials and devices.

In one aspect, the invention generally relates to a medical device for facilitating or causing hemostasis, comprising a matrix material of biocompatible carboxymethylcellulose having or adapted to have a plurality of open and interconnected cells, wherein the biocompatible carboxymethylcellulose is characterized by a degree of fabric substitution from about 0.2 to about 3.0, an average degree of polymerization from about 50 to about 2,000, and a carbonyl amount greater than 0 and below about 2% by weight of the total weight of the biocompatible carboxymethylcellulose.

In another aspect, the invention generally relates to a medical device for creating or enhancing tissue barrier, comprising a matrix material of biocompatible carboxymethylcellulose having or adapted to have a plurality of open and interconnected cells, wherein the biocompatible carboxymethylcellulose is characterized by a degree of fabric substitution from about 0.2 to about 3.0, an average degree of polymerization from about 50 to about 2,000, and a carbonyl amount greater than 0 and below about 2% by weight of the total weight of the biocompatible carboxymethylcellulose.

In yet another aspect, the invention generally relates to a medical device for facilitating or causing wound or burn healing, comprising a matrix material of biocompatible carboxymethylcellulose having or adapted to have a plurality of open and interconnected cells, wherein the biocompatible carboxymethylcellulose is characterized by a degree of fabric substitution from about 0.2 to about 3.0, an average degree of polymerization from about 50 to about 2,000, and a carbonyl amount greater than 0 and below about 2% by weight of the total weight of the biocompatible carboxymethylcellulose.

In yet another aspect, the invention generally relates to a medical device for facilitating or causing skin or tissue rejuvenation, comprising a matrix material of biocompatible carboxymethylcellulose having or adapted to have a plurality of open and interconnected cells, wherein the biocompatible carboxymethylcellulose is characterized by a degree of fabric substitution from about 0.2 to about 3.0, an average degree of polymerization from about 50 to about 2,000, and a carbonyl amount greater than 0 and below about 2% by weight of the total weight of the biocompatible carboxymethylcellulose.

In yet another aspect, the invention generally relates to a kit for wound, burn or cosmetic treatment, comprising a medical device of the invention.

In yet another aspect, the invention generally relates to a method for treating a hemostasis-related condition comprising applying a medical device of the invention to a patient at a wound site in need of hemostasis treatment.

In certain embodiments, the hemostasis-related condition relates to a surface bleeding or extremity arterial hemorrhage.

In yet another aspect, the invention generally relates to a method for creating a tissue barrier to treat an external or internal wound condition comprising applying a medical device of the invention to a patient at a wound or burn site in need of tissue barrier protection.

In yet another aspect, the invention generally relates to a method for treating a wound or burn-related condition comprising applying a medical device of the invention to a patient at a wound or burn site in need of healing facilitation.

In yet another aspect, the invention generally relates to a method for causing skin or tissue rejuvenation comprising applying a medical device of the invention to a patient at a skin or tissue site in need of rejuvenation treatment.

In yet another aspect, the invention generally relates to a method for making a matrix material of biocompatible carboxymethylcellulose. The method includes: purifying linter, wood and/or natural plant fiber by cooking and rinsing to afford extracted cotton pulp; crushing the extracted cotton pulp treating it NaOH and then CS₂ to make a viscous spinning solution; ejecting the spinning solution from a nozzle and through an acidic medium thereby solidifying it to form viscose fibers; cleaning the viscose fibers to remove residual chemicals; knitting the cleaned viscose fibers into woven fabrics; cleaning the woven fabrics; alkalizing the woven fabrics with a NaOH alkaline medium mixed with an alcohol to form alkalized woven fabrics; etherifying the alkalized woven fabrics; adjusting pH to be in the range from about 6 to about 8; and cleaning the woven fabrics.

### BRIEF DESCRIPTION OF THE DRAWINGS

FIG. 1 Hemostatic effect of biocompatible carboxymethylcellulose matrix (BCM) (a) and gel formation (b).

FIG. 2. Angiogram of animal treated with QuikClot Combat Gauze (CG) and BCM. The femoral artery was occluded at the injury site in 100% animals treated by CG (A: 2/2), and in 60% animals by BCM (B: 3/5). In 40% (C: 2/5) animals treated with BCM, the artery is narrowed at the injury site, but blood is present in the distal femoral artery. Analysis of the video angiogram demonstrated that this flow was antegrade, not retrograde from collateral circulation. Arrow indicates the artery injury site. Arrow head indicates blood flow at distal site away from artery injury location.

FIG. 3 Morphological assessments after tested materials removed at last step. After CG was removed from the wounds, hemostatic clot was ruptured and re-bleeding occurred (A), while a stable hemostatic clot and BCM formed sticky gel over the site of arterial injury was noted in BCM group (B).

FIG. 4. BCM observations at three time points. A: 45-second free bleed; B: 2-minute compression; C: 30-minute observation.

FIG. 5. Gross performance of skin contusion model. The BCM (low panel) reserved moisture than the control group (up-panel) and promote skin regeneration.

FIG. 6. HE staining of skin contusion model. There is no obvious scar were formed in both control and BCM group, while the thickness of skin in BCM group (low panel) is better than control group (middle panel).

FIG. 7. Gross performance of partial-thickness skin burn model: BCM promotes burn healing and skin regeneration.

FIG. 8. HE staining of partial-thickness skin burn model.

FIG. 9. Application of BCM on transplanted site during skin grafting (a). Wound area post tangential excisions; (b). Skin grafting on wound area, (c). Application of BCM over the wound bed.

FIG. 10. Application of BCM on donor site during skin grafting. (a). Wound area after taking skin graft; (b). BCM on wound area and stop bleeding in 30 second.

FIG. 11. BCM promoted grafted skin regeneration 14 days post transplantation. (a). Control (*Granulation tissue); (b). BCM treated. (Arrowhead: residual of BCM on the surface of healed wound).

FIG. 12. BCM promoted grafted skin regeneration 21 days post transplantation. (a). Control (*Granulation tissue); (b). BCM treated. (Arrowhead: residual of BCM on the surface of healed wound).

FIG. 13. BCM decreased wound surface bleeding on donor site 7 days post surgery. (a)&(b), Control; (c)&(d), BCM treated group.

FIG. 14. After 3 rounds of application of BCM on the left side of scalp, this side shows less exudation and hemorrhage, and minimal epithelial progression compared to the contralateral side treated with Bovine Collagen Silver Matrix.

### DESCRIPTION OF THE INVENTION

The invention provides a novel and significantly improved hemostasis, tissue barriers, wound and burn healing, and regenerative cosmetic materials and devices, which are made of water-soluble biocompatible carboxymethylcellulose matrix (BCM). The invention employs water-soluble cellulose hemostatic materials for the preparation of devices, articles, compositions and preparations. The compositions, devices and methods of the invention are applicable to internal and external hemostatic, internal and external wound healing, internal and external tissue barrier articles, and external cosmetic articles and compositions.

In one aspect, the invention generally relates to a medical device for facilitating or causing hemostasis. The medical device comprises a matrix material of biocompatible carboxymethylcellulose having or adapted to have a plurality of open and interconnected cells.

The biocompatible carboxymethylcellulose suitable for use in the present invention is characterized by (1) a degree of fabric substitution ranging from about 0.2 to about 3.0, (2) an average degree of polymerization from about 50 to about 2,000, and (3) a carbonyl amount greater than 0 and below about 2% by weight of the total weight of the biocompatible carboxymethylcellulose.

In another aspect, the invention generally relates to a medical device for creating or enhancing tissue barrier. The medical device comprises a matrix material of biocompatible carboxymethylcellulose having or adapted to have a plurality of open and interconnected cells. The biocompatible carboxymethylcellulose is characterized by (1) a degree of fabric substitution from about 0.2 to about 3.0, (2) an average degree of polymerization from about 50 to about 2,000, and (3) a carbonyl amount greater than 0 and below about 2% by weight of the total weight of the biocompatible carboxymethylcellulose.

In yet another aspect, the invention generally relates to a medical device for facilitating or causing wound or burn healing. The medical device comprises a matrix material of biocompatible carboxymethylcellulose adapted to have a plurality of open and interconnected cells. The biocompatible carboxymethylcellulose is characterized by (1) a degree of fabric substitution from about 0.2 to about 3.0, (2) an average degree of polymerization from about 50 to about 2,000, and (3) a carbonyl amount greater than 0 and below about 2% by weight of the total weight of the biocompatible carboxymethylcellulose.

In yet another aspect, the invention generally relates to a medical device for facilitating or causing skin or tissue rejuvenation. The medical device comprises a matrix material of biocompatible carboxymethylcellulose adapted to have a plurality of open and interconnected cells. The biocompatible carboxymethylcellulose is characterized by (1) a degree of fabric substitution from about 0.2 to about 3.0, (2) an average degree of polymerization from about 50 to about 2,000, (3) and a carbonyl amount greater than 0 and below about 2% by weight of the total weight of the biocompatible carboxymethylcellulose.

First, the biocompatible carboxymethylcellulose that may be employed in the present invention is characterized by a degree of fabric substitution ranging from about 0.2 to about 3.0, for example, from about 0.2 to about 2.5, from about 0.2 to about 2.0, from about 0.2 to about 1.5, from about 0.2 to about 1.2, from about 0.2 to about 1.0, from about 0.2 to about 0.8, from about 0.4 to about 3.0, from about 0.8 to about 3.0, from about 1.0 to about 3.0, from about 1.5 to about 3.0, from about 2.0 to about 3.0, from about 0.4 to about 2.5, from about 0.4 to about 2.0, from about 0.4 to about 1.5, from about 0.4 to about 1.2, from about 0.6 to about 2.5, from about 0.6 to about 2.0, from about 0.2 to about 0.9.

Second, the biocompatible carboxymethylcellulose that may be employed in the present invention is characterized by an average degree of polymerization from about 50 to about 2,000, for example, from about 50 to about 1,500, from about 50 to about 1,000, from about 50 to about 800, from about 50 to about 500, from about 100 to about 2,000, from about 200 to about 2,000, from about 500 to about 2,000, from about 1,000 to about 2,000, from about 100 to about 1,500, from about 100 to about 1,000, from about 100 to about 800, from about 100 to about 550.

Third, the biocompatible carboxymethylcellulose that may be employed in the present invention is characterized by a carbonyl amount greater than 0 and below about 2%, for example, below about 1.8%, below about 1.5%, below about 1.2%, below about 1.0%, below about 0.8%, below about 0.5%, and greater than 0%, by weight of the total weight of the biocompatible carboxymethylcellulose.

In certain embodiments, the matrix material comprises one or more salts selected from sodium salts, potassium salts, calcium salts, magnesium salts and aluminum salts.

In certain embodiments, the fabric substitution range is from about 0.2 to about 0.9 (e.g., 0.2, 0.3, 0.4, 0.5, 0.6, 0.7, 0.8, 0.9), and the degree of polymerization is from about 100 to about 550 (e.g., from about 100 to about 450, from about 100 to about 350, from about 100 to about 250, from about 150 to about 550, from about 200 to about 550, from about 250 to about 550, from about 150 to about 450, from about 150 to about 350).

In certain embodiments, the fabric substitution range is from about 0.45 to about 0.8, and the degree of polymerization is from about 150 to about 350.

In certain embodiments, the biocompatible carboxymethylcellulose is characterized by a pH from about 6 to about 8 (e.g., about 6.0, 6.5, 7.0, 7.5, 8.0), a chloride content equal to or less than about 10.0% (e.g., equal to or less than about 9%, 8%, 7%, 6%, 5%, 4%, 3%, 2%, 1%, and equal to or greater than 0%, 0.5%, 1%), and a sodium content in the range from about 6.5% to about 9.5% (e.g., about 6.5%, 7.0%, 7.5%, 8.0%, 8.5%, 9.0%, 9.5%).

In certain embodiments, the matrix material is in a form selected from powders, fibers, webs, nonwoven cloths, sponges, films, capsules, pellets, columns, plugs and colloids. In certain embodiments, the matrix material is in a form of powders. In certain embodiments, the matrix material is in a form of fibers. In certain embodiments, the matrix material is in a form of webs. In certain embodiments, the matrix material is in a form of nonwoven cloths. In certain embodiments, the matrix material is in a form of sponges. In certain embodiments, the matrix material is in a form of films. In certain embodiments, the matrix material is in a form of capsules. In certain embodiments, the matrix material is in a form of pellets. In certain embodiments, the matrix material is in a form of columns. In certain embodiments, the matrix material is in a form of plugs. In certain embodiments, the matrix material is in a form of colloids.

In yet another aspect, the invention generally relates to a kit for wound, burn or cosmetic treatment, comprising a medical device of the invention.

In certain embodiments, the kit is useful for wound healing. In certain embodiments, the kit is useful for burn healing. In certain embodiments, the kit is useful for cosmetic treatment.

In yet another aspect, the invention generally relates to a method for treating a hemostasis-related condition comprising applying a medical device of the invention to a patient at a wound site in need of hemostasis treatment.

In certain embodiments, the hemostasis-related condition relates to a surface bleeding or extremity arterial hemorrhage. In certain embodiments, the hemostasis-related condition comprises a surface bleeding. In certain embodiments, the hemostasis-related condition comprises an extremity arterial hemorrhage.

In yet another aspect, the invention generally relates to a method for creating a tissue barrier to treat an external or internal wound condition comprising applying a medical device of the invention to a patient at a wound or burn site in need of tissue barrier protection. In certain embodiments, the medical device of the invention is applied to a patient at a wound site in need of tissue barrier protection. In certain embodiments, the medical device of the invention is applied to a patient at a burn site in need of tissue barrier protection.

In certain embodiments, the external or internal wound condition relates to an arterial hemorrhage. In certain embodiments, the external or internal wound condition relates to a surface injury and bleeding.

In yet another aspect, the invention generally relates to a method for treating a wound or burn-related condition comprising applying a medical device of the invention to a patient at a wound or burn site in need of healing facilitation.

In certain embodiments, the medical device promotes cell proliferation and differentiation thereby healing in skin contusion and burn.

In yet another aspect, the invention generally relates to a method for causing skin or tissue rejuvenation comprising applying a medical device of the invention to a patient at a skin or tissue site in need of rejuvenation treatment.

In yet another aspect, the invention generally relates to a method for making a matrix material of biocompatible carboxymethylcellulose. The method includes: purifying linter, wood and/or natural plant fiber by cooking and rinsing to afford extracted cotton pulp; crushing the extracted cotton pulp treating it NaOH and then CS₂ to make a viscous spinning solution; ejecting the spinning solution from a nozzle and through an acidic medium thereby solidifying it to form viscose fibers; cleaning the viscose fibers to remove residual chemicals; knitting the cleaned viscose fibers into woven fabrics; cleaning the woven fabrics; alkalizing the woven fabrics with a NaOH alkaline medium mixed with an alcohol to form alkalized woven fabrics; etherifying the alkalized woven fabrics; adjusting pH to be in the range from about 6 to about 8; and cleaning the woven fabrics.

In certain embodiments, the matrix material of biocompatible carboxymethylcellulose produced by the disclosed method is characterized by a degree of fabric substitution from about 0.2 to about 3.0, an average degree of polymerization from about 50 to about 2,000, and a carbonyl amount greater than 0 and below about 2% by weight of the total weight of the biocompatible carboxymethylcellulose.

First, the biocompatible carboxymethylcellulose produced by the disclosed method is characterized by a degree of fabric substitution ranging from about 0.2 to about 3.0, for example, from about 0.2 to about 2.5, from about 0.2 to about 2.0, from about 0.2 to about 1.5, from about 0.2 to about 1.2, from about 0.2 to about 1.0, from about 0.2 to about 0.8, from about 0.4 to about 3.0, from about 0.8 to about 3.0, from about 1.0 to about 3.0, from about 1.5 to about 3.0, from about 2.0 to about 3.0, from about 0.4 to about 2.5, from about 0.4 to about 2.0, from about 0.4 to about 1.5, from about 0.4 to about 1.2, from about 0.6 to about 2.5, from about 0.6 to about 2.0, from about 0.2 to about 0.9.

Second, the biocompatible carboxymethylcellulose produced by the disclosed method is characterized by an average degree of polymerization from about 50 to about 2,000, for example, from about 50 to about 1,500, from about 50 to about 1,000, from about 50 to about 800, from about 50 to about 500, from about 100 to about 2,000, from about 200 to about 2,000, from about 500 to about 2,000, from about 1,000 to about 2,000, from about 100 to about 1,500, from about 100 to about 1,000, from about 100 to about 800, from about 100 to about 550.

Third, the biocompatible carboxymethylcellulose produced by the disclosed method is characterized by a carbonyl amount greater than 0 and below about 2%, for example, below about 1.8%, below about 1.5%, below about 1.2%, below about 1.0%, below about 0.8%, below about 0.5%, and greater than 0%, by weight of the total weight of the biocompatible carboxymethylcellulose.

In certain embodiments, the biocompatible carboxymethylcellulose produced by the disclosed method is characterized by a degree of fabric substitution ranging from about 0.2 to about 0.9 (e.g., 0.2, 0.3, 0.4, 0.5, 0.6, 0.7, 0.8, 0.9), and a degree of polymerization from about 100 to about 550 (e.g., from about 100 to about 450, from about 100 to about 350, from about 100 to about 250, from about 150 to about 550, from about 200 to about 550, from about 250 to about 550, from about 150 to about 450, from about 150 to about 350).

In certain embodiments, the biocompatible carboxymethylcellulose produced by the disclosed method is characterized by a degree of fabric substitution ranging from 0.45 to about 0.8, and a degree of polymerization is from about 150 to about 350.

In certain embodiments, the biocompatible carboxymethylcellulose produced by the disclosed method is characterized by a pH from about 6 to about 8 (e.g., about 6.0, 6.5, 7.0, 7.5, 8.0), a chloride content equal to or less than about 10.0% (e.g., equal to or less than about 9%, 8%, 7%, 6%, 5%, 4%, 3%, 2%, 1%, and equal to or greater than 0%, 0.5%, 1%), and a sodium content in the range from about 6.5% to about 9.5% (e.g., about 6.5%, 7.0%, 7.5%, 8.0%, 8.5%, 9.0%, 9.5%).

### EXAMPLES

### Example 1

A high-purity extract were purified from linter, wood and other natural plant fiber refining by cooking and rinsing, which were used in the manufacture of cellulose ethers. The purified cotton pulp was crushed by sodium hydroxide to make viscous spinning solution. The spinning solution is ejected from the nozzle through the acidic medium solidified to form viscose fibers. The viscose fiber were cleaned to remove residual chemicals and made into fabric woven. Then, the fabrics woven were placed in a reactor to react with sodium hydroxide alkaline medium med with alcohol. Alkalization treatment and etherification processes were conducted. After pH was modified to about 6 to about 8, fabric surface were cleaned of impurities with an alcoholic medium. The fabric substitution range is from about 0.45 to about 0.8, degree of polymerization is from about 150 to about 350. The finished indicators are pH6-8, chloride content≤10.0% and sodium content from about 6.5% to about 9.5%.

### Example 2

The water-soluble hydroxyethylcellulose could be prepared by the following processes: a). immersing the cellulose into an about 18% NaOH solution in an organic solvent (such as acetone, isopropanol, ortert-butylalcohol) at about 20-30° C. and alkalizing for 1 to 2 hours; 2). adding ethyleneoxide having a weight 1 to 1.5 times of the weight of the raw materials and allowing to react at 70-90° C. for 1 to 3 hours, neutralizing to pH 6 to pH 8 with an in organic acid (such as glacialacetic acid); 3). washing the resulting product with 70% to 90% organic solvent (V/V)(such as acetone, orethanol) aqueous solution; 4) dehydrating and drying (if required, freezing the product with liquid nitrogen and crushing into powders using a crusher).

### Example 3

The water-soluble etherized cellulose material (11 type) having a carbonyl content not greater than 2% and a degree of polymerization of 100-400 may be, for example, prepared by the following methods: a) using regenerated cellulose fabrics, fibers, powders, non woven cloths or sponges as raw materials; b) putting said raw materials into a closed reactor and allowing to react in a 2-3 g/L soft water solution of active chlorine (bath ratio 1:15-30) at pH 9-10 and at room temperature with stirring for 30-90 minutes, discharging, and Washing; c) reacting in a 2-3 g/L of hydrogen peroxide hard water solution in the presence of 1-5 g/L of a stabilizer at pH9-10 and a temperature of 80-100° C. With stirring for 50-60 minutes, washing with hot water. The following steps are identical to steps b); c); d); e); and f) in type 1 reaction.

### Example 4

Fifty gram of a viscose fabric was placed into a reactor, 1,000 mL of 2 g/L sodium hypochlorite was added to the reactor, pH was adjusted to 9-10.5, the materials were allowed to react at room temperature for 0.5-2 hours, drained, and washed with water, and then the pH was adjusted to 9.5-10.5. 2-4 g of a stabilizer (such as sodium silicate, sodium pyrophosphate or commercial hydrogen peroxide) and 1,000 mL of 25-30% hydrogen peroxide aqueous solution were added and the system was allowed to react at 85-100° C. With stirring for 1-2 hours, the resulting product was washed with hot Water of greater than 85° C. for three times. 150-200 mL of 30-70% chloroacetic acid solution in ethanol (W/W) was added to the reactor and the system was allowed to react at 20-30° C. With stirring for 1-2 hours, then 80-120 mL of 40-50% NaOH (W/W) aqueous solution and 280-320 mL of 95% (v/v) ethanol were added to the reactor and the system was allowed to continuously react at 20-75° C. for 1.5-5 hours. The resulting product was neutralized With 36% HCl (W/W) to pH 6-8 and washed with an ethanol solution having an ethanol content greater than 75% until the amount of Cl′ was less than 1%, dehydrated, dried, pack aged and sterilized to give type II oxidized carboxymethyl cellulose sodium fabric capable of being absorbed in vivo, which has a degree of substitution of 0.65-0.90 and a degree of polymerization less than 400.

### Example 5: Hemostatic Effect of BCM in Skin Cutting

To test the hemostatic effect of BCM in skin cutting and contusion, a total 8 swine were used in this experiment. Animals were housed on-site with enrichment and quarantined for at least four days for acclimation prior to experimentation. Swine were fasted for at least 12 hours but with free access to water before surgery. All anesthesia procedure were performed and maintained. These animals were randomly divided into 2 groups: (1) BCM treated group (4 animals); (2) CG treated group (4 animals). Two experimental hemostatic materials were tested in this study: 2″×2″ 2 layers CG (Z-MEDICA, LLC, Wallingford, Conn.) and 2″×2″, 2 layers of BCM (LifeScience PLUS, Inc., Mountain View, Calif.). A standardized skin cutting was made on the left side of abdomen (1″×1″). The tested materials were put onto the injury and pressed with continues pressure for 1 mins. The hemostatic effects were assayed within 3 mins.

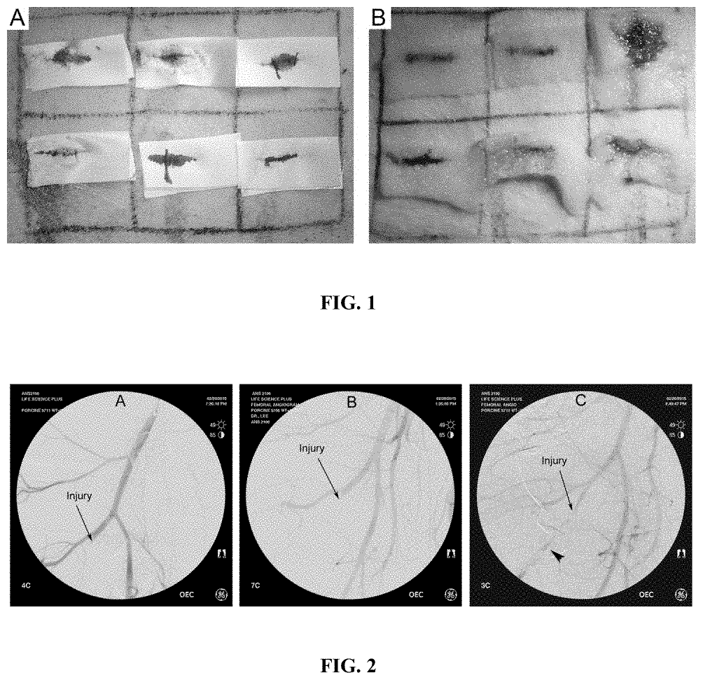

The result indicated the time to achieve hemostatic effect by BCM and CG was 1.2±0.34 min and 2.2±0.45 min respectively. And, BCM generated a gel on the top of injury site (FIG. 1).

### Example 6: Hemostatic Effect of BCM for Extremity Arterial Hemorrhage

This preclinical study was conducted at a GLP compliant laboratory (PMI Lab, San Carlos, Calif.). The protocol utilized was previously validated by the US Army Institute of Surgical Research and FDA for assessing bleeding control in large animal models. The experimental design and surgical procedures were assessed and cleared by the appropriate IACUC. Twelve healthy, male or female, Yorkshire cross-bred swine, weighing 33-47 kg, were purchased from Pork Power Farms (Turlock, Calif.) and used in all procedures. Animals were housed on-site with enrichment and quarantined for at least four days for acclimation prior to experimentation. Swine were fasted for at least 12 hours but with free access to water before surgery. All anesthesia procedure were performed and maintained by member from PMI lab. These animals were randomly divided into 2 groups: 1) BCM treated group (5 animals); 2) CG treated group (3 animals). Two experimental hemostatic materials were tested in this study: 3″×144″ Z-folded, 48 layers CG (, Z-MEDICA, LLC, Wallingford, Conn.) and 3″×24″, Z-folded, 8 layers of BCM (LifeScience PLUS, Inc., Mountain View, Calif.).

In order to determine the efficacy of a currently marketed novel hemostatic product biocompatible carboxymethylcellulose matrix (BCM) for severe bleeding control, a preclinical, large animal, pilot study was conducted. BCM is a new generation hemostat made from water soluble, oxidized etherized regenerated cellulose. It is a fully biocompatible, non-irritating, woven matrix of fibers that contains plant components. A validated protocol that has been accepted by food and drug administration (FDA) and the United States military was used on the surgery of the swine. In this pilot study, BCM demonstrated efficacy in several parameters: time of achieving initial hemostasis and post-treatment blood loss.

The baseline physiologic and hematological measurements among the treatment groups, including CG and BCM are similar (Table 1).

TABLE 1
Baseline Physiological and Hematological Measurements in Pigs

| Measurement | BCM | CG |
|---|---|---|
| BW(kg) | 38.38 ± 0.8 | 41.27 ± 2.49 |
| Temp (C. °) | 36.16 ± 0.6 | 37.07 ± 0.12 |
| MAP (mmHg) | 82.6 ± 15 | 68.33 ± 2.89 |
| HGB (g/dL) | 9.22 ± 0.7 | 8.067 ± 1.16 |
| HCT (%) | 28.92 ± 2.5 | 26.2 ± 4.07 |
| PLT (1000/ul) | 322.4 ± 80 | 288.3 ± 64.9 |
| PT (s) | 14.24 ± 0.4 | 14.3 ± 0.35 |
| aPTTs(s) | 13.94 ± 1.4 | 15.87 ± 0.21 |
| Fibrinogen (mg/dL) | 142 ± 27 | 138 ± 3.61 |
| Lactate (mM) | 15.12 ± 3.8 | 25.43 ± 1.75 |

In general, two treatments were required to produce hemostasis for all the products, including CG where hemostasis was not achieved even after three dressing application in one animal. BCM produced immediate hemostasis in one animal with one application, in two animals with two applications, and in two animals with three applications. CG could not produce hemostasis with three applications in one animal, and the pig died at 124 minutes due to continue bleeding. The simulated walking condition (movement of the legs) at the conclusion of experiments did not cause re-bleeding in surviving animals in both BCM and CG groups. The average times that bleeding was controlled by the dressings (Total time bleeding stopped) and other hemostatic outcomes are shown in Table 2. BCM controlled bleeding for 177.4±1.3 min, which is ˜50% longer than that achieved in CG (118.6±102 min).

The average pretreatment blood loss for all the animals was 6.71±1.91 mL/kg in BCM and 10.19±3.6 mL/kg in CG group (Table 2). The post treatment blood loss was 12.32±7.9 ml/kg in BCM and 16.1±25.5 ml/kg in CG group. Average blood loss in BCM group was nearly ⅔ of the CG groups.

For the survival time and rate, animals were monitored up to 3 hours or until death as determined by tidal PCO2<15 mm Hg or MAP<20 mm Hg, the survival time were calculated from the artery injury to 3 hours or until time of death. Animals in BCM group survived longer time than those in CG groups (Table 2).

TABLE 2
Outcomes of Treating a Groin Arterial Hemorrhage
with Different Hemostatic Dressings in Swine

| Outcome | BCM | | CG | |
|---|---|---|---|---|
| Initial hemostasis achieved (no | 1/5 | (11) | 2/3 | (5) |
| application | | | | |
| Total time bleeding stopped (min) | 177.4 ± 1.3 | | 118.6 ± 102 | |
| Pretreatment blood loss (mL/kg) | 6.71 ± 1.91 | | 10.19 ± 3.6 | |
| Posttreatment blood loss (mL/kg) | 12.32 ± 7.9 | | 16.1 ± 25.5 | |
| Total resuscitation fluid (mL/kg) | 98.3 ± 78.8 | | 79.3 ± 50.7 | |
| Survival rate | 5/5 | (100) | 3/2 | (66.7) |

| | | |
|---|---|---|
| Survival time (min) | 180 ± 0 | 161.31 ± 32 |

+Initial hemostasis was considered to occur when bleeding was stopped for at least 3 minutes after compression.

### Example 7: Barrier Effect of BCM for Arterial Hemorrhage

To check the arterial blood flow and vascular structures in the injured legs in both BCM and CG group, fluoroscopic angiography was performed through the cannulated right carotid artery. A catheter was guided down the aorta to the bifurcation, an angiogram was performed and images for treated and contralateral legs were recorded. The angiogram images of surviving animals showed complete blockage of blood flow in femoral arteries at the treated site by CG, while two animals from BCM groups shown partial blockage of blood flow in femoral arteries at the treated site by BCM, and blood flow could go through the injury site to the distal (FIG. 2).

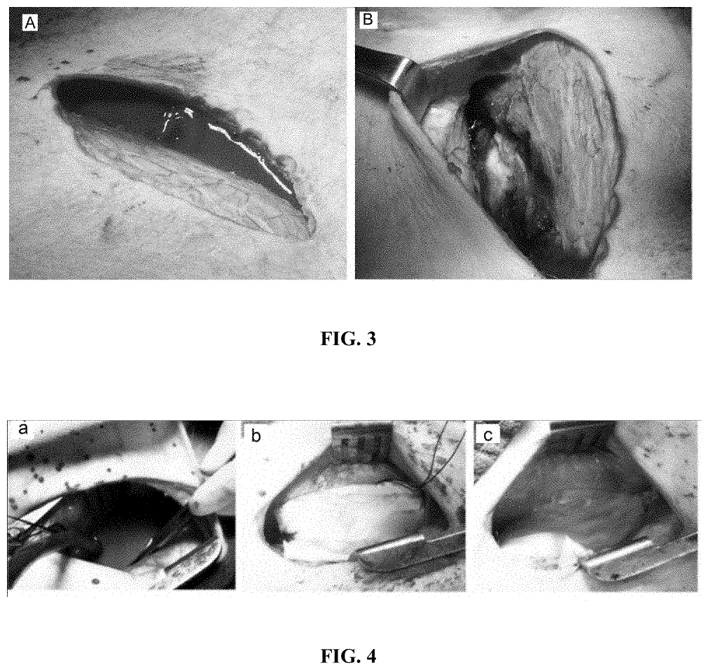

As observed, BCM did not absorb large quantities of blood. When in contact with blood, BCM formed an adherent gel that adhered to and served to create a safe and effective “seal membrane” over the site of injury, while CG dressings were easily removed from the wounds resulting in the rupture of the hemostatic clot and re-bleeding occurred at the final morphological assessment after tested materials removed. There is a significant difference on this point between the BCM and CG group, which is the most advantage of BCM compared to CG. At conclusion of experiments, CG dressings were easily removed from the wounds resulting in the rupture of the hemostatic clot and re-bleeding at the injury site in surviving animals (FIG. 3A). After removing the top layers of BCM, there were very strong, solid, and stable hemostatic clot and BCM formed an adherent gel over the site of arterial injury, which adhered to the site of injury and surrounding tissue. This adherent material served to create a safe and effective “seal membrane” (FIG. 3B).

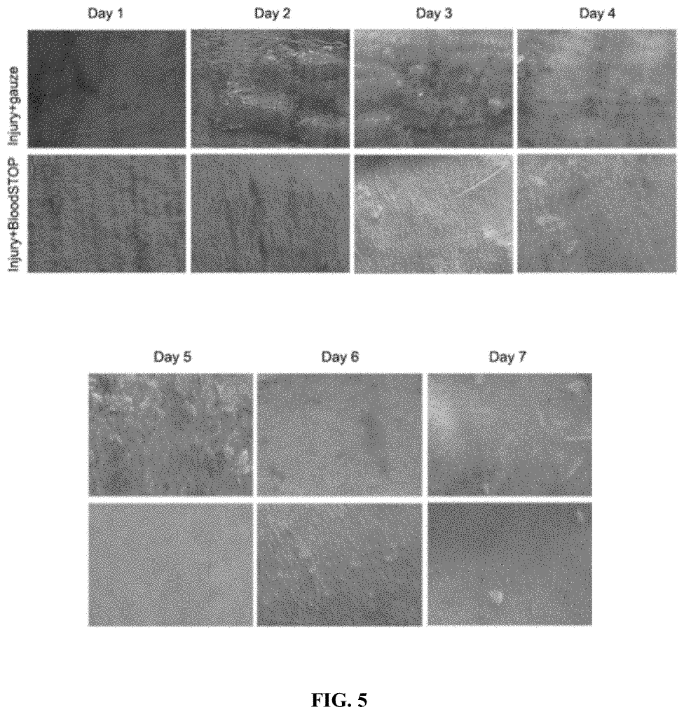

It was observed that BCM was sufficiently robust and it remained adherent to the injury site when the laparotomy sponges were removed from the wound. The combination of BCM and the clotting proteins creates a “seal membrane” that is highly stable to mechanical perturbation. This property will allow evacuation of the injured warrior without disruption of the clot (FIG. 4). The robustness of the clot will also allow for more measured surgical treatment at higher echelons of care. Surgeons will be able to confidently remove packing dressing material without fear of clot dislodgement and exsanguination in the operating room.

### Example 8: BCM Promote Skin Healing for Burn Wounds

A product made according to the present invention, Biocompatible carboxymethylcellulose matrix (or BCM, LifeScience PLUS, Mountain View, Calif., USA) is a biocompatible, woven fiber matrix made from regenerated cotton cellulose. In this study, when BCM contacted burn wound with liquid, BCM adhered to the wound and transferred to gel, stopped bleeding, and formed a protective layer, which resulted in the creation of an optimal wound healing environment.

A pre-clinical animal experiment and clinical trial to check the efficacy and safety of BCM was compared to standard Vaseline gauze, a commercial product widely used in the burn wound management.

### Part I. Pre-Clinical Animal Experiment

### Materials and Methods

Two burn related injury animal models were used in the pre-clinical experiment, including Contusion and Burn. A total 12 rabbit (male/female; 2.0-2.5 kg) were used in this initial experiment. Two-time points were used: 1 week and 2 weeks. Six rabbits underwent skin contusion and six partial-thickness skin burn.

For skin contusion model, the rabbit was shaved on the bilateral back. Skin contusion (20×80 mm, 0.2 mm thickness) was made using a file brush. Immediately after modeling, the injury site was covered with saline-soaked gauze. The injury sites were randomly divided into part A and part B. Part A was dressed with 2 layers of control substances (Vaseline gauze). Part B was dressed with 2 layers of BCM. Both areas were covered with gauze, which was sutured to the skin. The injury sites were observed and photos were taken daily. The dresses were changed daily from day 2. At 1 week and two weeks post the surgery, pathologic changes and scarring were checked with gross anatomy and histological assay.

For partial-thickness burn model, the rabbit was shaved on the bilateral back. A burn injury (20×80 mm, 0.2 mm thickness) was made using a 100° C. water bag for 8 sec. Immediately after modeling, the injury site was covered with saline-soaked gauze. The injury sites were randomly divided into part A and part B. Part A was dressed with 2 layers of control substances (Vaseline gauze). Part B was dressed with 2 layers of BCM. Both areas were covered with gauze, which was sutured to the skin. The injury sites were observed and photos were taken daily. The dresses were changed daily from day 2. At 1 week and two weeks post the surgery, pathologic changes and scarring were checked with gross anatomy and histological assay.

### Skin Contusion Model

The control group, scabbing happened one day after contusion; Redness and swelling last at least five days. For the BCM group, no significant scarring and swelling were observed; three days later, the appearance returned to normal (FIG. 5).

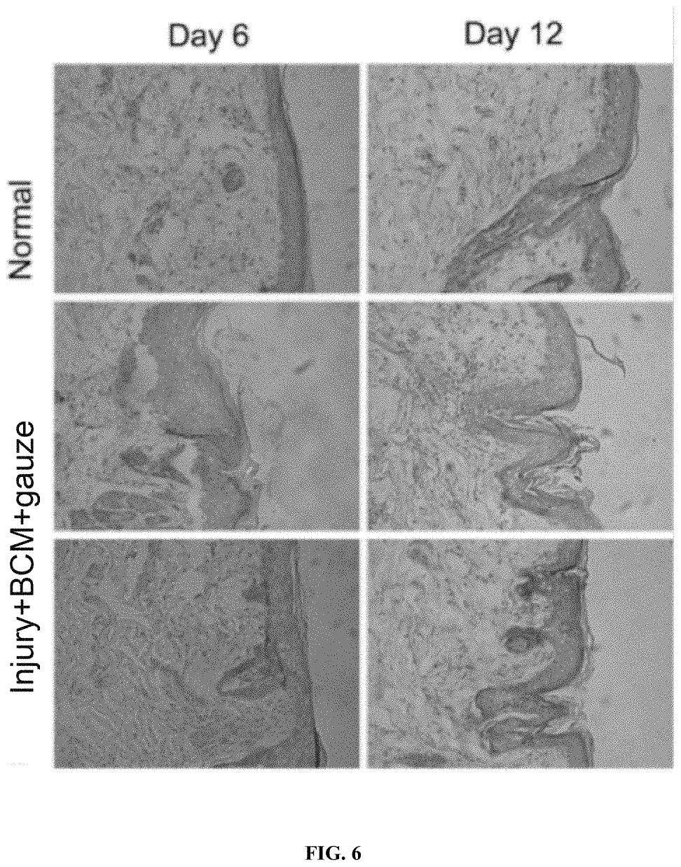

After 6 days, HE staining found that most of the injury site in the control group had returned to normal. However, some areas still showed excoriation. In the BCM group, it was found that the pathological changed completely back to normal. After 12 days, abnormalities were not found in both the control group and the BCM group (FIG. 6).

### Partial-Thickness Skin Burn Model

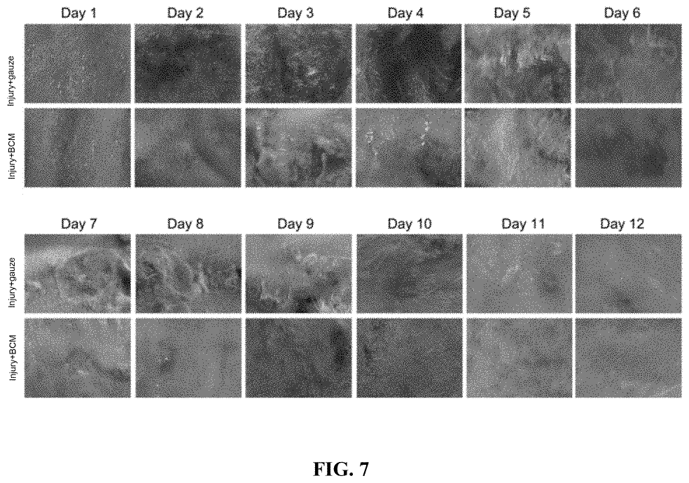

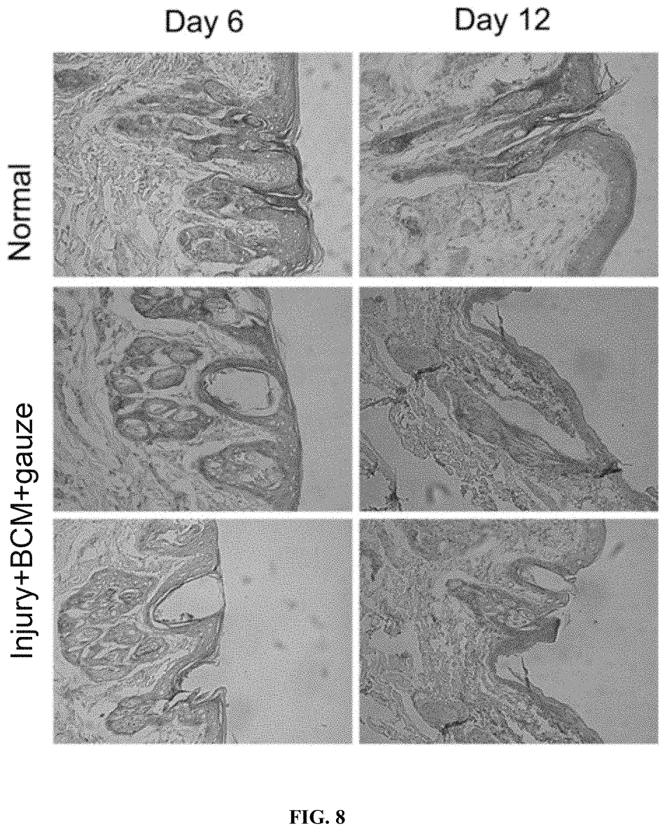

In the control group, the most common symptoms were redness and swelling at 1 day after modeling. Small blisters and scarring were observed with the naked eyes from the next day. Significant skin damage lasted for at least ten days. Twelve days later, the appearance returned to normal. In BCM group, significant scarring and swelling were also observed. However, the appearance returned to normal from the eighth day (FIG. 7). HE staining showed that typical blisters formed at the sixth day and disappeared at the twelfth day in the control group. In the BCM group, it was also found that blisters formed at day 6. However, the skin damage in the BCM group was slighter than that in the control group in the corresponding time point. After 12 days, blisters were also not found in the BCM group (FIG. 8).

These results demonstrated that BCM can significantly repair pathological changes both in the skin contusion model and partial-thickness skin burn model and can be used for healing skin damages.

### Part II Clinical Trial: Safety and Effective of BCM for Burn Wound Care

### Materials and Methods

Upon the outcome of pre-clinical experiment, an informal clinical trail was conduced following the protocol below.

As per this protocol, BCM was used in non-infected burn wounds post the acute stage, especially for the skin graft transplantation used after tangential excision of burns to decrease blood loss at the donor site (DS) and aid in providing a moist wound environment and enhance tissue healing at the transplanted site (TS). After cleaning of burn wounds or after tangential debridement of burn wounds the product were applied on both DS and TS. The patients were on appropriate antibiotic coverage dependent on their conditions, as the BCM does not contain antimicrobial treatment. Wounds covered with BCM continued to produce exudate and would be moist while careful monitoring of the site(s) was/were done. As per institutional guidelines, dressing sites were kept clean or sterile and monitored, and dressing changes were performed by the appropriate certified practitioners.

In this Stage-I initial informal clinical trail, a total 6 patients were recruited, including 4 patients treated with BCM on both DS and TS site and 2 patients treated without BCM as Control. The treatment procedure was followed according to the Clinical Therapeutic Protocol #2, #3, #4, and #5 below.

### Clinical Therapeutic Protocol

Debridement of wounds, tangential excisions and skin grafting were done at the discretion of the practitioner with sedation or anesthesia as per policy. Once clean wounds were obtained application of BCM was done.

### 1. Debridement of Burn Wounds Non-Tangential Excisions

1) After wounds were debridement to the best ability BCM in 2 layers were placed over open wounds that were not in pressure bearing or dependent areas.
2) After this, placement of nonstick dressing, for example, Telfa, Adaptic, or a reasonable facsimile was done.
3) Then wrapping with gauze bandage roll was recommended with the possible placement of additional overlying dressings as per the practitioner's discretion.
4) In areas of pressure where the patient was in a supine position, for example, the posterior aspect of the head, the elbows, or similar areas, a different technique were used. In these areas 4-6 layers of BCM were used followed by the appropriate nonstick dressing and wrap.
5) It is recommended that dressings be changed daily or every other day based on the exudate production and the judgment of the practitioner.

### 2. Intraoperative Burn Debridement, Tangential Excisions, and Skin Grafting

1) During intraoperative treatment of burns where bleeding is a concern, BCM may be placed.
2) After creation of the appropriate wound bed by debridement, BCM may be placed initially as a double layer to help decrease bleeding.
3) After the skin graft was obtained, BCM may be placed initially as a double layer to help decrease bleeding on the donor site (DS).
4) With the placement of this initial double layer pressure should be maintained.
5) After 2-5 minutes of pressure and attaining adequate hemostasis additional layers of BCM should be placed.
6) For skin grafting: after skin graft stamp were transplanted to wound bed area post-tangential excisions, the BCM were placed above the skin grafts.
7) Multiple layers may be appropriate secondary to the exudative property of the wound site, amount of bleeding, wound location, and condition of the patient. Current guideline for non-dependent (non weight bearing) sites is the application of 1-2 layers in addition to the initial double layer placed for hemostasis.
8) Additional layers are recommended in wounds with significant blood loss or significant exudate or dependent positions.
9) Once all layers of BCM have been placed there should be placement of nonstick dressing for example: Telfa, Adaptic, or a reasonable facsimile followed by placement of gauze bandage roll.
10) After placement of gauze bandage role practitioners may decide on additional reinforcing dressings.
11) A modification in technique which is appropriate for more extensive areas with bleeding, is placement of BCM in 2-4 layers on top of a nonstick dressing on top of gauze and applying this “sandwich” to the area being treated. This will allow for pressure to be applied to the area of concern without disruption of the wound site and ability to wrap the site without disturbing the BCM. Therefore there will be no disruption of the hemostasis attained with the dressing.

### 3. Post BCM Placement Care

1) During the post-debridement, post BCM placement period, wound should be monitored according to practitioner standard of care.
2) During this time if significant bleeding is still noted intervention as per the practitioner is appropriate.
3) During the post-placement period if significant exudate is noted reinforcement of the dressings as per the practitioner is appropriate.
4) As per practitioner protocol and institutional protocol patient should have vital signs monitored as their condition warrants.
5) Practitioners should be notified of any change in the dressings as per usual policy and procedure.

### 4. Reapplication of BCM

1) In patients with burns and severe wounds there was need to reapply BCM. This was done several ways depending on the conditions and location of the dressing change. As conditions allow BCM was changed under sterile or clean conditions as per the practitioner.
2) The preferred technique was removal of outer dressings to the level of BCM. Depending on the bleeding of the wound or the exudative level there was some BCM present on the wound.
3) When additional tangential debridement was required then proceed as above. When wounds appear clean facilitated removal of BCM with Saline or Sterile Water.
4) The BCM became gel. Wipe away gel. It was not necessary to wipe all gel away as long as wounds are noted to be clean.
5) With clean and stable wounds reapply the BCM as outlined above taking care to place additional layers on pressure bearing areas, such as posterior scalp, elbows, heels, etc.
6) The more frequent the dressing changes the fewer layers of BCM was required (in non-weight bearing areas). For example a forearm burn/wound may require one layer of BCM with nonstick dressing and wrap, as long as it was monitored daily.

### 5. Special Sites

Application to the face was possible. Care was taken to ensure that the gel created from the BCM did not enter the eyes or interfere with the patient's airway. Periorbital, perinasal, and perioral application was done at the discretion of the practitioner.

### Application of BCM on Skin Graft Transplanted Site

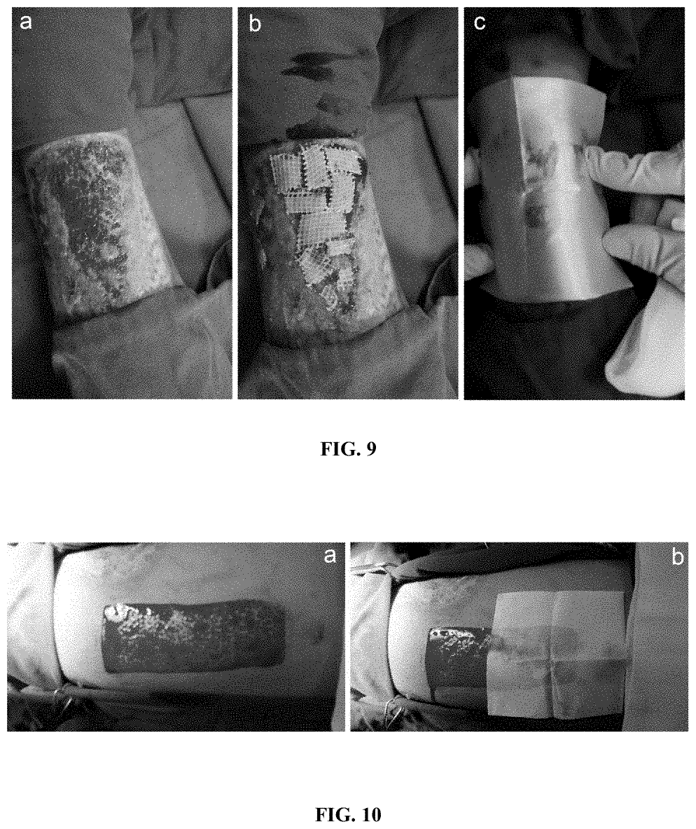

Post the extensive tangential excisions, the stamp-like skin grafts were transplanted on the fresh surface of wound area where minor bleeding continued. The BCM were directly applied above the skin grafts and wound area with continue pressure for 3 min. until BCM transformed into a gel, and then Telfa and regular gaze were applied to form a “sandwich” dressing (FIG. 9).

### Application of BCM on Donor Site During Skin Grafting

During skin grafting, after skin was taken from the donor site, fresh wound were formed and bleeding occurred immediately. The BCM was applied onto the wound surface to stop bleeding (FIG. 10) followed by Telfa and regular gaze to form a “sandwich” dressing. Bleeding was stopped in 30 sec. post the application of BCM.

### BCM Enhanced Tissue Healing Post Skin Grafting

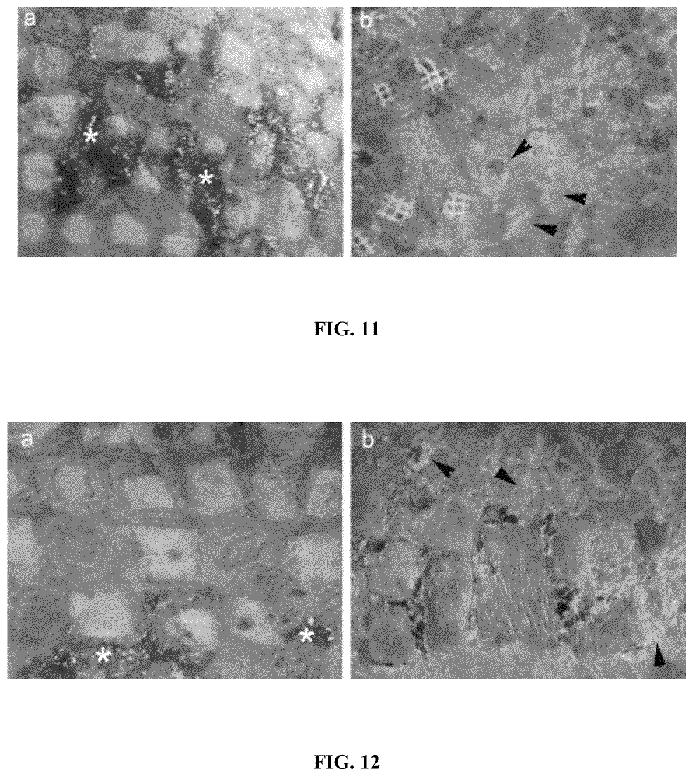

In patients treated with BCM, the BCM became a gel overlapped on the transplanted skin graft and anchored them in situ. Importantly, BCM promoted skin cell proliferation and regeneration. At 14 days post the transplantation, the entire wound area was covered by new skin, while in the control group, there was still 40% wound area were granulation tissue (FIG. 11).

Strikingly, 21 days post skin graft transplant, there was still 10% area of granulation tissue in the control group, while a mature skin was formed in BCM treated group (FIG. 12).

### Hemostatic Effect of BCM on Donor Site

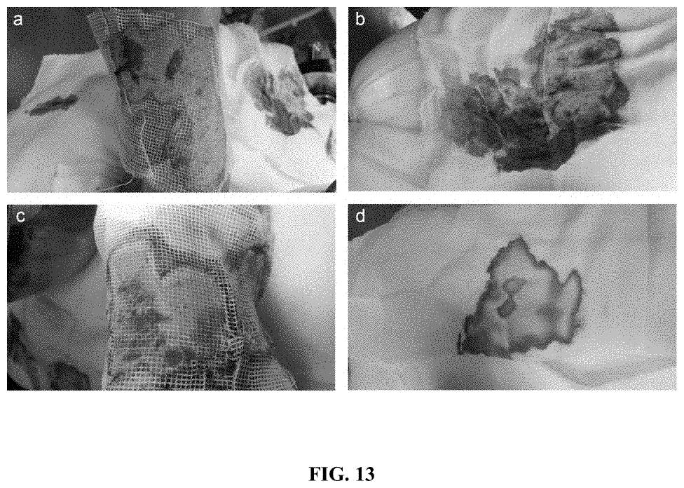

The hemostatic effect on skin graft donor site was very significant, which achieved 30 sec. post application on fresh wound surface and the effect lasted for 7 days (FIG. 13).

Based on these clinical trail results, it was evident that BCM was safe and effective for burn wound care for skin grafting on both donor site and transplanted site. BCM benefited skin grafting by anchoring transplanted skin graft in-situ, promoting skin regenerating and tissue healing, and stopping bleeding on donor site.

### Example 9: BCM in the Management of Dermal Erosions in a Patient with Hay-Wells Syndrome

Hay-Wells syndrome is an autosomal dominant disorder. Clinically, children with this disorder present with erythroderma and erosions, especially of the scalp. Treatment is focused on skin care. Gentle wound care with bland emollients and silicone-based dressings is recommended but usually with unsatisfactory outcome. The use of cellulose-based gauze is well established in battlefield wound but is uncommon in dermal defect. A 9-year-old girl presented with scalp, thigh and chest dermal defect due to this syndrome was admitted to the hospital. She had been given debridement surgeries and dermal transplantation surgery and many forms of hemostasis agents but with unsatisfactory clinical outcomes. The use of BCM enabled satisfactory hemostatic, anti-infection, and pro-tissue regeneration effects. BCM facilitated the repair of large defects and avoided increased risk for infection associated skin defects. This example supports the use of BCM in dermal erosions.

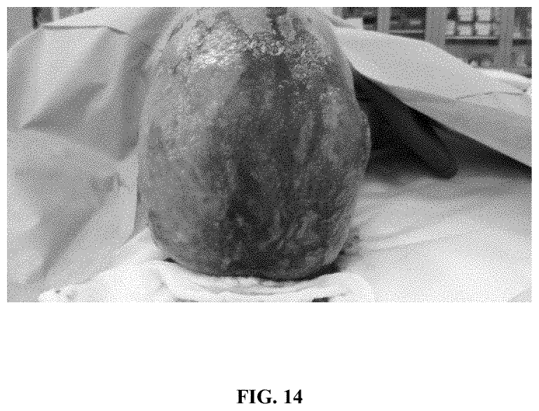

A 9-year old female patient, born from non-consanguineous parents was referred to the clinic due to skin lesions. At admission, she presented dermal erosions covered with honey-colored hematic crusts on the thigh and chest especially on the scalp. The dermal lesions also extended to auricles of both sides. A biocompatible, non-irritating, hemostatic agent (BCM) which resembles traditional gauze on the scalp and topical antibiotics in areas with erosions and exudation was initiated. After 3 rounds of application of BCM on the left side of scalp, this side showed less exudation and hemorrhage and minimal epithelial progression comparing to the contralateral side treated with Bovine Collagen Silver Matr (FIG. 14). In the meantime, green mucus appeared on the right side of scalp with cultures indicating Pseudomonas aeruginosa infection. Therefore, systemic Vancomycin treatment was started and applied BCM to the right side of scalp.

### Example 10: Cosmetic Effect of BCM Solution-Carboxymethylcellulose Serum

A carboxymethylcellulose serum was made by dissolve BCM in ddH2O at 0.01˜8% (wt/v). The key to dissolving BCM is to add the solid carefully to the water so that it is well dispersed (well-wetted) then adding more water followed. Adding water to the dry solid produces a “clump” of solid that is very difficult to dissolve; the solid must be added to the water. Stir gently or shake intermittently for 24 hours at room temperature; do not stir constantly with a magnetic stirring bar. High heat is not needed and may actually slow down the solubilization process. Under normal conditions, the effect of temperature on solutions of this product is reversible, so slight temperature variation has no permanent effect on viscosity. This product is a high viscosity carboxymethylcellulose (CMC); the viscosity of a 1% solution in water at 25° C. is 1500-3000 centipoise (cps). The viscosity is both concentration and temperature dependent. As the temperature increases, the viscosity decreases. As the concentration increases, the viscosity increases. Low, medium and high viscosity CMCs are all used as suspending agents. Low viscosity CMC is usually used in “thin” aqueous solutions.

Carboxymethylcellulose serum is hydrating and lubricating gel-like substance. It binds with water to add plumpness to the skin. Carboxymethylcellulose serum soak skin in lush moisture, supporting youthful plumpness and a smooth, even complexion. To open the stratum corneum of skin, chemical or mechanical methods will be used in cosmetic field. After opening of stratum corneum, BMC will be applied to the surface of skin, where it will become a gel and some molecules will be infused into the subcutaneous space to improve wrinkle and promote new skin cells regeneration.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the following description, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1-27. (canceled)

28. A method for treating a skin burn comprising applying to a patient at a burn site a medical device comprising
a matrix material of biocompatible carboxymethylcellulose having a plurality of open and interconnected cells, wherein the biocompatible carboxymethylcellulose is characterized by
a degree of fabric substitution from about 0.45 to about 0.8, an average degree of polymerization from about 150 to about 350,
a pH from about 6 to about 8, a chloride content equal to less than about 10.0%, and
a sodium content in the range from about 6.5% to about 9.5%, in each case by weight of the total weight of the biocompatible carboxymethylcellulose, and
bioactive agents that stimulate healing of skin burn and prevent or reduce infection,
wherein the medical device promotes cell proliferation and differentiation thereby healing the skin burn.

29. The method of claim 28, wherein the medical device further comprises one or more anti-fibrinolysis agents and one or more coagulation factors.

30. The method of claim 28, wherein the matrix material is in a form selected from powders, fibers, webs, nonwoven cloths, sponges, films, capsules, pellets, columns, plugs and colloids.
